(19) United States
(12) Patent Application Publication
(10) Pub. No.: US 2024/0182389 A1
(43) Pub. Date: Jun. 6, 2024

(54) PROPYLENE POLYOL CONVERSION TO OLEFIN MONOMER

(71) Applicant: Basell Poliolefine Italia S.r.l., Milan (IT)

(72) Inventors: Sandor Nagy, Seabrook, TX (US); David S. Mannel, Humble, TX (US); Natalia Nagy, Houston, TX (US); Amy D. Rose, Humble, TX (US); George R. Horvat, Baytown, TX (US); Larry W. Arndt, Humble, TX (US); David W. Leyshon, Houston, TX (US); Xueyong Yang, Bellaire, TX (US); Barbara Kimmich, Houston, TX (US)

(73) Assignee: Basell Poliolefine Italia S.r.l., Milan (IT)

(21) Appl. No.: 18/526,362

(22) Filed: Dec. 1, 2023

Related U.S. Application Data
(60) Provisional application No. 63/385,890, filed on Dec. 2, 2022.

(51) Int. Cl. C07C 29/60; C07C 1/24; C07C 29/80

(52) U.S. Cl. CPC C07C 29/60; C07C 1/24; C07C 29/80

(57) ABSTRACT

Processes for conversion of propylene polyol feed into useful petrochemical products, including olefin monomers, are described. Such processes comprise: adding a feed stream comprising one or more propylene polyols, hydrogen, and optionally water, to a catalytic conversion reaction zone in the presence of a first solid acid catalyst component and a hydrogenation catalyst component and reacting at a pressure and temperature sufficient to form a first product stream comprising a propanol component. The first product stream can be added to a dehydration reaction zone in the presence of a dehydration catalyst and reacted at a pressure and temperature sufficient to form a second product stream comprising propylene.

### CROSS-REFERENCE TO RELATED APPLICATIONS

The application claims the benefit of priority to U.S. Provisional Patent Application No. 63/385,890, filed on Dec. 2, 2022, which is incorporated herein by reference in its entirety.

### FIELD OF THE DISCLOSURE

The disclosure relates to methods of catalytically converting propylene glycols into propylene and/or propylene precursors. Methods for conversion of propylene glycols (or intermediate products) are simultaneously reacted with a dehydration cleavage catalyst component and a hydrogenation catalyst component, and optionally thereafter with a dehydration catalyst.

### BACKGROUND OF THE DISCLOSURE

Propylene glycol substances are found in waste streams and/or by-products associated with a number of industrial processes. Such waste streams and by-products can include a number other constituents and in some cases a significant amount of water. Significant sources of propylene glycols occur during recycling of certain post-consumer waste oxygen-containing plastics, are produced as waste streams during resulting from the production of propylene oxides, and are a by-product of aircraft de-icing and other industrial activities where products containing propylene glycol are used as an anti-freeze agent. Such propylene glycol substances are miscible in water and exhibit a low potential to volatilize from water or soil in both pure and dissolved forms. Propylene glycol exerts high levels of biochemical oxygen demand during degradation in surface waters.

Heightened standards of living and increased urbanization have led to an increased demand for polymer products, including, but not limited to, polyurethane (“PU”) and polyethylene terephthalate (“PET”). PET is the most common thermoplastic polymer resin of the polyester family and is used in fibers for clothing, containers for liquids and foods, thermoforming for manufacturing, and in combination with glass fiber for engineering resins. The downside to the demand for polymer products is the increase in waste. Post-consumer plastic waste typically ends up in landfills, with about 12% being incinerated and about 9% being diverted to recycling. In landfills, most plastics do not degrade quickly, becoming a major source of waste that overburdens the landfill. Incineration is also not an ideal solution to treating the plastic wastes as incineration leads to the formation of carbon dioxide and other greenhouse gas emissions. As such, there has been much interest in developing methods of recycling plastic waste to reduce the burden on landfills while being environmentally friendly. Post-consumer waste comprising PU and PET can be degraded by a glycolysis reaction with propylene glycols in the presence of trans-esterification catalysts, resulting in a glycolic component comprising propylene glycols (mono-, di-, and/or higher propylene glycols), which may further include butane-diol, ethylene glycols, and other oxygenated components.

One process for production of propylene oxide, known as PO/TBA, involves the co-oxidation of isobutylene, producing propylene oxide and tertiary butyl alcohol. Another process for production of propylene oxide, known as POSM, involves the co-oxidation of ethyl benzene, producing propylene oxide and styrene monomer. These processes produce waste streams comprising propylene glycols (mono-, di-, and/or higher propylene glycols.

Deicing fluids come in a variety of types and are typically composed of ethylene glycol (EG) or propylene glycol (PG), along with other ingredients. Propylene glycol-based fluid is more common because it is less toxic than ethylene glycol. Deicing a large commercial aircraft produces significant amounts of diluted propylene glycol waste fluid. Propylene glycol is also used as an anti-freeze fluid in geothermal wells where leakage of propylene glycol can affect dissolved oxygen on ground and surface water.

There is a continued need for the development of a robust processes for the conversion of propylene glycols to higher value and/or environmentally desirable dispositions. Ideally, such processes would be highly flexible and could be implemented with commonly used equipment and familiar techniques to produce a wide variety of products.

### SUMMARY OF THE DISCLOSURE

The present disclosure provides methods for catalytic conversion of propylene polyols to higher value products useful as feedstocks to other processes. In some embodiments, a process comprises adding a feed stream comprising one or more propylene polyols, hydrogen, and optionally water, to a catalytic conversion reaction zone in the presence of a catalytic conversion catalyst to form a first reaction mixture. The first reaction mixture is reacted under temperature and pressure conditions sufficient to produce a catalytic conversion product stream comprising a propanol component. In some embodiments, the catalytic conversion product stream can be fed to a dehydration reaction zone in the presence of a dehydration catalyst to form a second reaction mixture. Alternatively, in some embodiments, the catalytic conversion product stream can be sent to a first distillation column to produce a catalytic conversion product overhead stream and a catalytic conversion product bottoms stream. The first distillation column is a fractioning or distillation column and includes equipment associated with the column, such as heat exchangers, decanters, pumps, compressors, valves, and the like. The catalytic conversion product overhead stream can be fed to the dehydration reaction zone in the presence of a dehydration catalyst to form an alternate second reaction mixture.

In some embodiments, the second reaction mixture or the alternate second reaction mixture is reacted under temperature and pressure conditions sufficient to produce a dehydration product stream comprising propylene for further processing. Alternatively, in some embodiments, the dehydration product stream can be sent to a second distillation column to produce a dehydration product overhead stream comprising propylene and a dehydration product bottoms stream comprising water. The second distillation column is a fractioning or distillation column and includes equipment associated with the column, such as heat exchangers, decanters, pumps, compressors, valves, and the like.

In some embodiments, the process further comprises adding an organic waste stream, comprising one or more propylene polyols and a first content of one or more impurities harmful to the dehydration cleavage catalyst component and/or a hydrogenation catalyst component, to a guard reaction zone to form the feed stream comprising one or more propylene polyols.

The foregoing has outlined rather broadly the features and technical advantages of the present disclosure in order that the detailed description of the disclosure that follows may be better understood. Additional features and advantages of the disclosure will be described hereinafter, which form the subject matter of the claims of the disclosure. It should be appreciated by those skilled in the art that the conception and specific embodiments disclosed may be readily utilized as a basis for modifying or designing other catalyst compositions and/or processes for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1A is a simplified flow diagram of the disclosed process having a catalytic conversion reaction zone, according to embodiments of the disclosure;

FIG. 1B is a simplified flow diagram of the dehydration reaction zone, optionally used in conjunction with the flow shown in FIG. 1A, according to embodiments of the disclosure;

FIG. 1C is a simplified flow diagram of the guard reaction zone, optionally used in conjunction with the flow shown in FIG. 1A or the combination of FIG. 1A and FIG. 1B, according to embodiments of the disclosure;

FIG. 2A is a simplified flow diagram of the disclosed process having a catalytic conversion reaction zone followed by a first distillation column, according to embodiments of the disclosure;

FIG. 2B is a simplified flow diagram of the dehydration reaction zone followed by a second distillation column, optionally used in conjunction with the flow shown in FIG. 2A, according to embodiments of the disclosure; and

FIG. 2C is a simplified flow diagram of the guard reaction zone, optionally used in conjunction with the flow shown in FIG. 2A or the combination of FIG. 2A and FIG. 2B, according to embodiments of the disclosure.

While the disclosed process and composition are susceptible to various modifications and alternative forms, the drawings illustrate specific embodiments herein described in detail by way of example. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims.

### DETAILED DESCRIPTION OF THE DISCLOSURE

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase. It must also be noted that, as used in the specification and the appended claims, the singular forms “a,” “an,” and “the” include plural references unless otherwise specified.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “propylene glycol,” in reference to the feed stream to the process disclosed herein, refers to mono-propylene glycol, di-propylene glycol, tri-propylene glycol, tetra-propylene glycol, higher polypropylene glycols, or a combination thereof.

As used herein, “impurities,” in reference to the organic waste stream, refers to material present that can reduce the abilities of dehydration cleavage catalyst component and a hydrogenation catalyst component to perform catalytic conversion of the propylene glycols in the feed to the catalytic conversion reaction zone. In some embodiments, impurities comprise amines, urethane, amides, other nitrogen containing hydrocarbons, organic bases, caustic, or a combination thereof.

As used herein, “post-consumer waste” refers to a type of waste produced by the end consumer of a material stream.

As used herein, “post-industrial waste” refers to a type of waste produced during the production process of a product.

As used herein, “reaction zone” refers to a chamber sufficiently enclosed to maintain selected operating conditions within the chamber to produce a desired reaction, such as a catalytic conversion reaction zone or a dehydration reaction zone. In some embodiments, each reaction zone can be a separate reactor. In some embodiments, a single vessel can contain a plurality of reaction zones.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in patent law, e.g., they allow for elements not explicitly recited, but exclude elements that are found in the prior art or that affect a basic or novel characteristic of the disclosure.

The term “pure” as used in reference to the feed stream refers to a feed that is 100% polyolefin, but does not mean that the feed contains only one type of polyolefin. Rather, a “pure” feed stream can have a mixture of polyolefins such as low-density polyethylene, high density polyethylene, polypropylene and combinations thereof.

All concentrations herein are by weight percent (“wt. %”) unless otherwise specified.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the disclosure.

The phrase “substantially all of” means greater than or equal to 95 wt %, greater than or equal to 99 wt %, greater than or equal to 99.5 wt %, or greater than or equal to 99.9 wt %.

The following abbreviations are used herein:

| ABBREVIATION | TERM |
|---|---|
| Beta | Beta (or BEA) zeolite |
| DPG | Di-propylene glycol |
| H-USY | H-ultra-stable zeolite Y |
| MPG | Mono-propylene glycol |
| PET | poly(ethylene terephthalate) |
| PPO | Propylene polyol |
| PU | Polyurethane |
| T4PG | Tetra-propylene glycol |
| TGA | Thermogravimetric Gravimetric Analysis |
| TPG | Tri-propylene glycol |
| USY | Ultra-stable zeolite Y |
| wt % | Weight percent |
| WHSV | Weight hourly space velocity |
| ZSM-5 | Zeolite Socony Mobil-5 |

### Catalytic Conversion of Propylene Polyols

The present disclosure provides catalytic conversion of propylene polyols to produce products comprising propylene and/or propylene precursors. In some embodiments, a process comprised adding a feed stream comprising one or more propylene glycols, hydrogen, and optionally water to a catalytic conversion reaction zone comprising a dehydration cleavage catalyst component and a hydrogenation catalyst component to form a first reaction mixture. The first reaction mixture is reacted under temperature and pressure conditions sufficient to form a catalytic conversion product comprising a propanol component. In some embodiments, at least a portion of the catalytic conversion product is recycled as a portion of the feed to the catalytic conversion reaction zone.

In some embodiments, the catalytic conversion product is sent as a feed stream to a dehydration reaction zone. Alternatively, the catalytic conversion product is sent to a first distillation column to form a catalytic conversion product overhead stream comprising a propanol component and a catalytic conversion product bottoms stream, and the catalytic conversion product overhead stream is sent as a feed stream to a dehydration reaction zone.

The catalytic conversion product, or alternatively the catalytic product overhead stream, are added to a dehydration zone comprising a dehydration catalyst to form a second reaction mixture. In the alternative embodiments where the catalytic conversion product bottoms stream is formed, at least a portion of the catalytic conversion product bottoms stream is recycled as additional feed to the catalytic conversion reaction zone. The second reaction mixture is reacted under temperature and pressure conditions sufficient to form a dehydration product comprising propylene.

In some embodiments, the dehydration product is sent to further processing for recovery of the propylene. Alternatively, the dehydration product is sent to a second distillation column to form a dehydration product overhead stream comprising propylene and a dehydration product bottoms stream comprising water, and the dehydration product overhead stream is sent to further processing for recovery of the propylene.

In some embodiments, the process further comprises adding an organic waste stream, comprising one or more propylene polyols and a first content of one or more impurities harmful to the dehydration cleavage catalyst component and/or the hydrogenation catalyst component, to a guard reaction zone to form the feed stream comprising one or more propylene polyols. In some embodiments, such impurities comprise amines, urethane, amides, other nitrogen containing hydrocarbons, organic bases, caustic, or a combination thereof. In some embodiments, the guard reaction zone comprises a reactive bed comprising generic absorbants, clays, diatomites, activated carbon, or a combination thereof. In some embodiments, the guard reaction zone can be operated at a pressure in the range of from 0 psig (0 kPag) to 10 psig (69 kPag) and a temperature in the range of from 20° C. to 30° C.

### Catalytic Conversion Reaction

The feed to the catalytic conversion reaction zone comprises one or more propylene polyols, hydrogen, and optionally water. In some embodiments, the hydrogen is added in an amount such that the molar ratio of hydrogen (H₂) to oxygen (O) in the polyol feed is in the range of from 1 to 20 or from 5 to 10. The catalytic conversion reaction zone comprises a dehydration cleavage catalyst component and, a hydrogenation catalyst component. In some embodiments, the one or more propylene polyols comprise propylene glycol, di-propylene glycol, tri-propylene glycol, tetra-propylene glycol, or a combination thereof. A first reaction mixture is formed from the feed and the catalyst when the feed is added to the reaction zone.

In some embodiments, the first reaction mixture is reacted at: a temperature in the range of from 20° C. to 600° C., from 50° ° C. to 500° C., or from 100° C. to 350° C.; a pressure in the range of from 100 psig (689 kPag) to 1,500 psig (10,340 kPag), 250 psig (1,724 kPag) to 1,250 psig (8,620 kPag), 350 psig (2,413 kPag) to 900 psig (6,205 kPag), from 500 psig (3,450 kPag) to 1,000 psig (6,890 kPag), or from 750 psig (5,171 kPag) to 1,100 psig (7,584 kPag), or a combination thereof.

In some embodiments, the one or more polyols and optionally water are added to the catalytic conversion reaction zone at a weight hourly space velocity in the range of from 0.1 h⁻¹ to 100 h⁻¹, 0.3 h⁻¹ to 50 h⁻¹, 0.5 h⁻¹ to 5 h⁻¹, 0.1 h⁻¹ to 3.0 h⁻¹, 0.02 h⁻¹ to 15 h⁻¹, 0.5 h⁻¹ to 5 h⁻¹, or 0.3 h⁻¹ to 1.0 h⁻¹.

The reaction produces a hydrogenation product stream comprising a propanol component. In some embodiments, the propanol component comprises n-propanol, iso-propanol, or a combination thereof. In some embodiments, the hydrogenation product stream comprises 0 wt % to 90 wt % water and 10 wt % to 100 wt % organics other than water. In some embodiments, the organics other than water comprise 1-propanol in the range of from 50 wt % to 90 wt % and other C₃ hydrocarbons in the range of from 10 wt % to 50 wt %, wherein weight percentages are based on the total weight of the organics other than water.

In some embodiments, the catalytic conversion product stream is fed to a first distillation column to produce a catalytic conversion product overhead stream comprising a propanol component and a catalytic conversion product bottoms stream. In some embodiments, the catalytic conversion product overhead stream is fed to a dehydration reaction zone, and the catalytic conversion product bottoms stream is recycled as additional feed to the catalytic conversion reaction zone.

### Catalytic Conversion Catalyst

In some embodiments, the catalytic conversion catalyst comprises a first solid acid catalyst component and a hydrogenation catalyst component.

In some embodiments, the solid acid catalyst component comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, one or more polystyrene sulfonate acidic resins, sulfonate functionalized support, tethered organic sulfonic acids, acidic clays, or a combination thereof.

In some embodiments, the first solid acid catalyst is further characterized by one or more of the following:
a) the zeolite component comprises zeolite-H-Y, zeolite-H-ZSM5, zeolite-H-beta, zeolite-H-mordenite, zeolite-H-ferrierite, or a combination thereof;
b) the alumina silicate comprises amorphous alumina silicate, acid washed layered alumina silicate, or a combination thereof, wherein in some embodiments, the acid washed layered alumina silicate comprises bentonite, vermiculite, or a combination thereof;
c) the aluminum phosphate comprises SAPO 34;
d) the supported phosphoric acid comprises a silica, clay, or alumina support;
e) the one or more supported tungsten oxides comprise a silica, clay, or alumina support;
f) the one or more tungsten oxides comprise tungsten oxide, tungsten di-oxide, tungsten tri-oxide, tungsten pentoxide, or a combination thereof;
g) the supported tungstosilicic acid comprises a silica, clay, or alumina support;
the supported phosphomolybdic acid comprises a silica, clay, or alumina support; and
the sulfonate functionalized support is silica.

Any one of the foregoing embodiments can be further characterized in that the hydrogenation catalyst comprises nickel (Ni), Raney Ni, cobalt (Co), molybdenum (Mo), ceria (Ce), magnesium (Mg), gold (Au), iridium (Ir), osmium (Os), palladium (Pd), platinum (Pt), rhodium (Rh), ruthenium (Ru), tungsten (W), titanium (Ti), NiMo, CoMo, NiW, CoW, Ru, Pt, Pd, or a combination thereof. In some embodiments, the foregoing metal or metals are supported on silica, alumina, silica alumina, zeolite, activated carbon, or a combination thereof. In some embodiments, the catalyst is sulfided prior to hydrogenation. In some embodiments, the hydrogenation catalyst comprises sulfided NiMo/Al₂O₃, sulfided CoMo/Al₂O₃, Ni/SiO₂, Ni/Al₂O₃, Raney Ni, Cu/SiO₂, Cu/Al₂O₃, Pd/SiO₂, Pd/Al₂O₃, Pd/C, Pt/SiO₂, Pt/Al₂O₃, Ru/C, In₂O₃ In₂O₃/Al₂O₃, In₂O₃/SiO₂, or a combination thereof.

In some embodiments, the first solid acid catalyst component is a discrete solid acid catalyst, and the hydrogenation catalyst component is a discrete hydrogenation catalyst. In some embodiments, a hybrid catalyst comprises the first solid acid catalyst component and the hydrogenation catalyst component.

### Dehydration Reaction

In some embodiments, the feed to the dehydration reaction zone comprises the hydrogenation product. In some embodiments, the hydrogenation product is sent to a distillation column to produce a hydrogenation product overhead stream and a hydrogenation product bottoms stream, and the hydrogenation product overhead stream is sent as feed to the hydrogenation reaction zone. A second reaction mixture is formed from the feed, and the catalyst when the feed are added to the hydrogenation reaction zone.

In some embodiments, the second reaction mixture is reacted at: a temperature in the range of from 20° ° C. to 600° C., from 50° ° C. to 400° C., or from 100° ° C. to 300° C.; a pressure in the range of from 15 psig (103 kPag) to 500 psig (689 kPag), from 100 psig (689 kPag) to 450 psig (3,100 kPag), or from 200 psig (1,379 kPag) to 400 psig (2,760 kPag); or a combination thereof.

In some embodiments, the hydrogenation product stream or the hydrogenation product overhead stream is added to the dehydration reaction zone at a weight hourly space velocity in the range of from 0.1 h⁻¹ to 100 h⁻¹, 0.5 h⁻¹ to 60 h⁻¹, or 0.8 h⁻¹ to 25 h⁻¹.

The reaction produces a dehydration product stream comprising propylene. In some embodiments, the dehydration product stream comprises propylene in the range of from 80 wt % to 100 wt % and other hydrocarbons in the range of from 0 wt % to 20 wt %, wherein weight percentages are based on the total weight of the dehydration product stream. In some embodiments, the other hydrocarbons comprise C₂, C₃, and/or C₄ oxygenates, C₂, C₃, and/or C₄ olefins, or a combination thereof.

In some embodiments, the dehydration product stream is sent to further processing and recovery of the propylene. In some embodiments, the dehydration product stream is fed to a second distillation column to produce a dehydration product overhead stream comprising the propylene and a dehydration product bottoms stream. In some embodiments, the dehydration product overhead stream is sent to further processing and recovery of the propylene.

### Dehydration Catalyst

In some embodiments, the dehydration catalyst comprises a second solid acid catalyst component. In some embodiments, the second solid catalyst component comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, or a combination thereof.

In some embodiments, the second solid acid catalyst is further characterized by one or more of the following:
a) the zeolite component comprises zeolite-H-Y, zeolite-H-ZSM5, zeolite-H-beta, zeolite-H-mordenite, zeolite-H-ferrierite, or a combination thereof;
b) the alumina silicate comprises amorphous alumina silicate, acid washed layered alumina silicate, or a combination thereof, wherein the acid washed layered alumina silicate comprises bentonite, vermiculite, or a combination thereof;
c) the aluminum phosphate comprises SAPO 34;
d) the supported phosphoric acid comprises a silica, clay, or alumina support;
e) the one or more supported tungsten oxides comprise a silica, clay, or alumina support; and
f) the one or more tungsten oxides comprise tungsten oxide, tungsten di-oxide, tungsten tri-oxide, tungsten pentoxide, or a combination thereof.

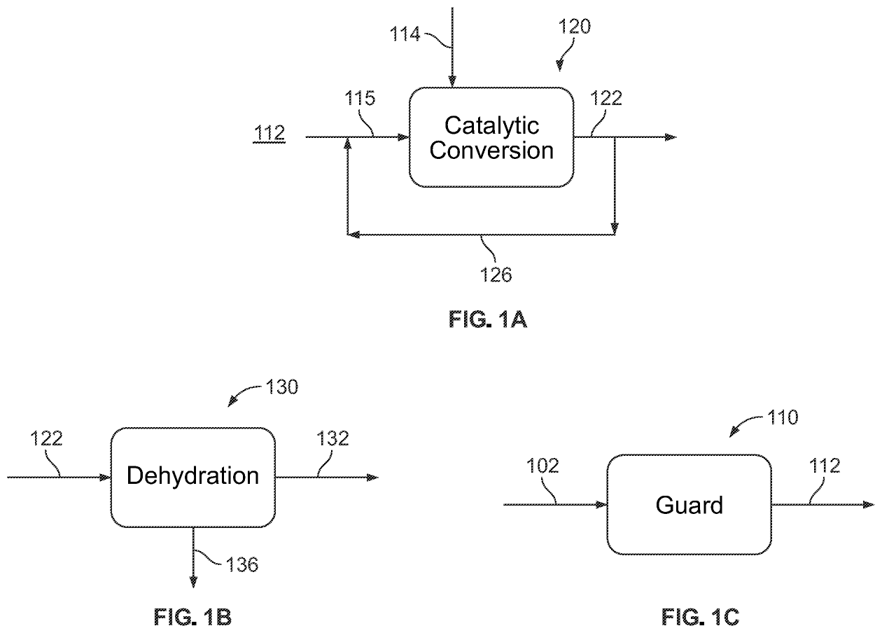

FIG. 1A-FIG. 1C show embodiments without a distillation column after each reaction zone. In FIG. 1A, a feed comprising one or more propylene glycols 112 and hydrogen 114 is obtained as feed to catalytic conversion reaction zone 120. In some embodiments, stream 126 is added with stream 112 to form the feed stream to the catalytic conversion reaction zone 120 comprising a first solid acid catalyst component and a hydrogenation catalyst component. These streams can be mixed prior to addition to catalytic conversion reaction zone 120 or alternatively added to catalytic conversion reaction zone 120 at different locations.

After the reaction in catalytic conversion reaction zone 120, catalytic conversion product 122 is withdrawn from catalytic conversion reaction zone 120. In some embodiments, a portion of catalytic conversion product 122 is sent as recycle stream 126 as additional feed to catalytic conversion reaction zone 120.

In some embodiments, as shown in FIG. 1B, hydrogenation product 122 is added as a feed stream to dehydration reaction zone 130. After the reaction in dehydration reaction zone 130, dehydration product 132 is withdrawn from dehydration reaction zone 130. Water 136 is also withdrawn from dehydration reaction zone 130 as a by-product of the dehydration reaction.

In some embodiments, as shown in FIG. 1C, an organic waste stream 102 containing impurities harmful to the first solid acid catalyst component and/or the hydrogenation catalyst component is added as a feed stream 102 to guard reaction zone 110. Guard reaction product 112 comprising on or more propylene glycols is withdrawn from guard reaction zone 110.

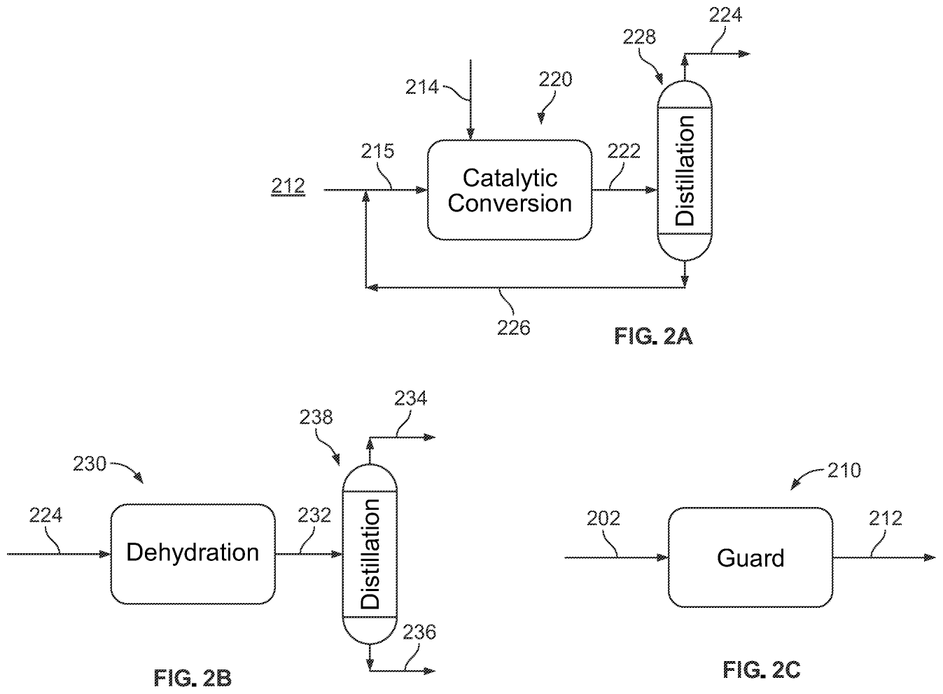

FIG. 2A-FIG. 2C show embodiments with a distillation column after the catalytic conversion reaction zone. In FIG. 2A, a feed comprising one or more propylene glycols 212 and hydrogen is obtained as feed to catalytic conversion reaction zone 220. In some embodiments, stream 226 is added with stream 212 to form the feed stream to the catalytic conversion reaction zone 220 comprising a first solid acid catalyst component and a hydrogenation catalyst component. These streams can be mixed prior to addition to catalytic conversion reaction zone 220 or alternatively added to catalytic conversion reaction zone 220 at different locations.

After the reaction in catalytic conversion reaction zone 220, catalytic conversion product 222 is withdrawn from catalytic conversion reaction zone 220 and added as a feed stream to distillation column 228 from which catalytic conversion product overhead stream 224 and catalytic conversion product bottoms stream 226 are withdrawn. In some embodiments, the catalytic conversion product bottoms stream 226 is sent as additional feed to catalytic conversion reaction zone 220.

As shown in FIG. 2B, hydrogenation product overhead stream 224 is added as a feed stream to dehydration reaction zone 230. After the reaction in dehydration reaction zone 230, dehydration product 232 is withdrawn from dehydration reaction zone 230 and added as a feed stream to distillation column 238 from which dehydration product overhead stream 234 and dehydration product bottoms stream 236 are withdrawn.

In some embodiments, as shown in FIG. 2C, an organic waste stream 202 containing impurities harmful to the first solid acid catalyst component and the hydrogenation catalyst component is added as a feed stream 202 to guard reaction zone 210. Guard reaction product 212 comprising on or more propylene glycols is withdrawn from guard reaction zone 210.

### Certain Embodiments

In some embodiments, the process comprises adding a feed stream comprising one or more propylene polyols, hydrogen, and optionally water, to a catalytic conversion reaction zone in the presence of a first solid acid catalyst component and a hydrogenation catalyst component to form a first reaction mixture. In some embodiments, the hydrogen is added in an amount such that the molar ratio of hydrogen (H₂) to oxygen (O) in the polyol feed is in the range of from 1 to 20 or from 5 to 10. In some embodiments, the one or more propylene polyols comprise propylene glycol, di-propylene glycol, tri-propylene glycol, tetra-propylene glycol, or a combination thereof.

In some embodiments, the first reaction mixture is reacted at: a temperature in the range of from 20° ° C. to 600° C., from 50° ° C. to 500° C., or from 100° ° C. to 350° C.; a pressure in the range of from 100 psig (689 kPag) to 1,500 psig (10,340 kPag), 250 psig (1,724 kPag) to 1,250 psig (8,620 kPag), 350 psig (2,413 kPag) to 900 psig (6,205 kPag), from 500 psig (3,450 kPag) to 1,000 psig (6,890 kPag), or from 750 psig (5,171 kPag) to 1,100 psig (7,584 kPag), or a combination thereof, to form a catalytic conversion product stream comprising a propanol component. In some embodiments, the propanol component comprises n-propanol, iso-propanol, or a combination thereof. In some embodiments, the dehydration product stream further comprises a propylene precursor component, wherein in some cases the propylene precursor component comprises 1 propanol, 2-propanol, propionaldehyde, acetone, C₃ dioxanes, C₃ dioxolanes, propylene glycol, hydroxyacetone, or a combination thereof.

In some embodiments, the feed stream is added to the catalytic conversion reaction zone at a weight hourly space velocity in the range of from 0.1 h⁻¹ to 100 h⁻¹, 0.3 h⁻¹ to 50 h⁻¹, or 0.5 h⁻¹ to 5 h⁻¹, 0.1 h⁻¹ to 3.0 h⁻¹, 0.02 h⁻¹ to 15 h⁻¹, 0.5 h⁻¹ to 5 h⁻¹, or 0.3 h⁻¹ to 1.0 h⁻¹, or any combination thereof.

In some embodiments, the process further comprises either:
a) withdrawing the catalytic conversion product stream as a first conversion product; or
b) feeding the catalytic conversion product to a first distillation column to produce a catalytic conversion product overhead stream and a catalytic conversion product bottoms stream, recovering the catalytic conversion product overhead stream comprising a propanol component, and withdrawing the catalytic conversion product overhead stream as a second conversion product.

In further embodiments at least a portion of the first conversion product or the second conversion product is added to the catalytic conversion reaction zone as additional feed.

In some embodiments, in addition to any of the foregoing, the process further comprises adding either the first conversion product or the second conversion product to a dehydration reaction zone in the presence of a dehydration catalyst to form a second reaction mixture. In either instance, the second reaction mixture is reacted at: a temperature in the range of from 20° C. to 600° C., from 50° ° C. to 400° C., or from 100° ° C. to 300° C.; a pressure in the range of from 15 psig (103 kPag) to 500 psig (689 kPag), from 100 psig (689 kPag) to 450 psig (3,100 kPag), from 200 psig (1,379 kPag) to 400 psig (2,760 kPag); or a combination thereof, to form a dehydration product comprising propylene.

In some embodiments, the dehydration product is fed to second distillation column to produce a dehydration product overhead stream comprising a propanol component and a dehydration product bottoms stream. In some embodiments, the dehydration product stream comprises propylene in the range of from 80 wt % to 100 wt % and other hydrocarbons in the range of from 0 wt % to 20 wt %, wherein weight percentages are based on the total weight of the dehydration product stream. In some embodiments, the other hydrocarbons comprise C₂, C₃, and/or C₄ oxygenates, C₂, C₃, and/or C₄ olefins, or a combination thereof.

In some embodiments, in addition to any one of the foregoing embodiments, the process further comprises adding an organic waste stream, comprising one or more propylene polyols and a first content of one or more impurities harmful to the first solid acid catalyst component and/or the hydrogenation catalyst component, to a guard reaction zone to form the feed stream comprising one or more propylene polyols.

Any one of the foregoing embodiments can be further characterized in that the dehydration cleavage catalyst component comprises a first solid acid catalyst. In some embodiments, the first solid acid catalyst component comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, one or more polystyrene sulfonate acidic resins, sulfonate functionalized support, tethered organic sulfonic acids, acidic clays, or a combination thereof. In some embodiments, the first solid acid catalyst is further characterized by one or more of the following:
a) the zeolite component comprises zeolite-H-Y, zeolite-H-ZSM5, zeolite-H-beta, zeolite-H-mordenite, zeolite-H-ferrierite, or a combination thereof;
b) the alumina silicate comprises amorphous alumina silicate, acid washed layered alumina silicate, or a combination thereof, wherein in some embodiments, the acid washed layered alumina silicate comprises bentonite, vermiculite, or a combination thereof;
d) the aluminum phosphate comprises SAPO 34;
e) the supported phosphoric acid comprises a silica, clay, or alumina support;
f) the one or more supported tungsten oxides comprise a silica, clay, or alumina support;
g) the one or more tungsten oxides comprise tungsten oxide, tungsten di-oxide, tungsten tri-oxide, tungsten pentoxide, or a combination thereof;
h) the supported tungstosilicic acid comprises a silica, clay, or alumina support;
i) the supported phosphomolybdic acid comprises a silica, clay, or alumina support; and
j) the sulfonate functionalized support is silica.

In some embodiments, the zeolite catalyst component of the first solid acid catalyst has a SiO₂/Al₂O₃ mole ratio of less than or equal to 200, less than or equal to 100, less than or equal to 50, less than or equal to 25, or less than or equal to 15. In some embodiments, the zeolite catalyst component has a SiO₂/Al₂O₃ mole ratio of greater than or equal to 0.5, greater than or equal to 1, greater than or equal to 3, greater than or equal to 5, or greater than or equal to 10. In some embodiments, the zeolite catalyst component has a SiO₂/Al₂O₃ mole ratio in the range of from 0.5 to 200, from 1 to 100, form 3 to 50, from 5 to 25, or from 10 to 15.

Any one of the foregoing embodiments can be further characterized in that the hydrogenation catalyst component comprises nickel (Ni), Raney Ni, cobalt (Co), molybdenum (Mo), ceria (Ce), magnesium (Mg), gold (Au), iridium (Ir), osmium (Os), palladium (Pd), platinum (Pt), rhodium (Rh), ruthenium (Ru), tungsten (W), titanium (Ti), NiMo, CoMo, NiW, CoW, Ru, Pt, Pd, or a combination thereof. In some embodiments, the foregoing metal or metals are supported on silica, alumina, silica alumina, zeolite, activated carbon, or a combination thereof. In some embodiments, the catalyst is sulfided prior to hydrogenation. In some embodiments, the hydrogenation catalyst comprises sulfided NiMo/Al₂O₃, sulfided CoMo/Al₂O₃, Ni/SiO₂, Ni/Al₂O₃, Raney Ni, Cu/SiO₂, Cu/Al₂O₃, Pd/SiO₂, Pd/Al₂O₃, Pd/C, Pt/SiO₂, Pt/Al₂O₃, Ru/C, In₂O₃ In₂O₃/Al₂O₃, In₂O₃/SiO₂, or a combination thereof.

In some embodiments, the first solid acid catalyst component is a first discrete catalyst, and the hydrogenation catalyst component is a second discrete catalyst. In some embodiments, a hybrid catalyst comprised the first solid acid catalyst component and the hydrogenation catalyst component.

Any one of the foregoing embodiments can be further characterized in that the dehydration catalyst comprises a second solid acid catalyst. In some embodiments, the second solid acid catalyst comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, or a combination thereof. In some embodiments, the second solid acid catalyst is further characterized by one or more of the following:
a) the zeolite component comprises zeolite-H-Y, zeolite-H-ZSM5, zeolite-H-beta, zeolite-H-mordenite, zeolite-H-ferrierite, or a combination thereof;
b) the alumina silicate comprises amorphous alumina silicate, acid washed layered alumina silicate, or a combination thereof, wherein the acid washed layered alumina silicate comprises bentonite, vermiculite, or a combination thereof;
c) the aluminum phosphate comprises SAPO 34;
d) the supported phosphoric acid comprises a silica, clay, or alumina support;
e) the one or more supported tungsten oxides comprise a silica, clay, or alumina support;
and
f) the one or more tungsten oxides comprise tungsten oxide, tungsten di-oxide, tungsten tri-oxide, tungsten pentoxide, or a combination thereof.

In some embodiments, the zeolite catalyst component of the second solid acid catalyst has a SiO₂/Al₂O₃ mole ratio of less than or equal to 200, less than or equal to 100, less than or equal to 50, less than or equal to 25, or less than or equal to 15. In some embodiments, the zeolite catalyst component has a SiO₂/Al₂O₃ mole ratio of greater than or equal to 0.5, greater than or equal to 1, greater than or equal to 3, greater than or equal to 5, or greater than or equal to 10. In some embodiments, the zeolite catalyst component has a SiO₂/Al₂O₃ mole ratio in the range of from 0.5 to 200, from 1 to 100, from 3 to 50, from 5 to 25, or from 10 to 15.

The presently disclosed methods for conversion of propylene polyols to propylene and/or propylene precursors are exemplified with respect to the examples below. These examples are included to demonstrate embodiments of the appended claims. However, these are exemplary only, and the disclosure can be broadly applied to any combination of propylene polyol feed, with and without water and/or impurities, and disclosed catalysts. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### Examples

The following examples are included to demonstrate embodiments of the disclosure. It should be appreciated by those of skill in the art that the techniques disclosed in the examples which follow represent techniques discovered to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Catalytic Conversion Process

Catalytic conversion reactions could be performed in a continuous feed packed bed reactor. The tube reactor consists of a 3 inch (7.6 cm) long ¼″ (6.4 mm) diameter stainless steel tube packed with 0.15 g of dehydration cleavage solid acid catalyst (such as CP 811 E-75 CY (1.6) (beta-zeolite extrudate)) and 0.15 g of hydrogenation catalyst (such as sulfided NiMo/Al₂O₃). The tube reactor could be operated under isothermal conditions maintained by an electric clam-shell furnace. Liquid feed of 90 wt % TPG and 10 wt % water could be fed using an ISCO 500D syringe pump along with hydrogen to achieve the desired WHSV 2.75 h⁻¹. Reactor pressure was set and maintained using a back pressure regulator A typical reaction pressures were 750 psig (5,070 kPag). Product compositions were analyzed by injecting the product stream on an Agilent 7890 GC equipped with a flame ionization detector (FID).

### Example(s) 1-2

Table 1 summarizes the results of Examples 1 and 2. Examples 1 and 2 were conducted in a tubular reactor using 3 grams of hydrogenation catalyst (a sulfided NiMo). In each Example, the liquid feed was a mixture of TPG and water. The liquid feed was fed to the reactor along with hydrogen (at 500 sccm and 600 psig) at a temperature of 600° C. Both examples achieved 100% conversion with a selectivity of 85-90% to a mixture of 1-propanol and 2-propanol.

TABLE 1

| | | | Catalyst | Liquid | H₂ | H₂ | | | Selectivity to |
|---|---|---|---|---|---|---|---|---|---|
| | | | Mass | Flow | Flow | Pressure | Temp. | | 1-propanol + |
| Ex. | Feed | Catalyst | (g) | (mL/min) | (sccm) | (psig) | (° C.) | Conversion | 2-propanol |
| 1 | 80 wt % TPG, | NiMo—S | 3 | 0.025 | 500 | 600 | 195 | 100% | 85-90% |
| | 20 wt % water | | | | | | | | |
| 2 | 60 wt % TPG, | NiMo—S | 3 | 0.025 | 500 | 600 | 195 | 100% | 80-85% |
| | 40% water | | | | | | | | |

### Dehydration Process

A catalytic conversion product produced using the above catalytic conversion product reaction conditions can be fed to a dehydration reaction zone comprising aluminum phosphate as a solid acid catalyst at a WHSV of 10 h⁻¹. A typical dehydration reaction can be conducted at a temperature of about 200° C. and a pressure of 300 psig (2,070 kPag) to produce a dehydration product comprising 80 wt % to 100 wt % propylene after removal of all water produced in the reaction.

### Example(s) 3-11

Table 2 summarizes the dehydration yields of 1-propanol to propylene at a temperature of 275° C. and a pressure of 300 psig. The Examples are based on a feed rate of 0.022 mL/min of an 80 wt % TPG, 20 wt % water feed. In each Example, 0.5 g of the specified catalyst was used.

TABLE 2

| | | Yield (%) | | | |
|---|---|---|---|---|---|
| Ex. | Catalyst | 1-Propanol | Propylene | Unknown | Others |
| 3 | Ferrierite ZD18018TL¹ | 0 | 94 | 5 | 1 |
| 4 | Bentonite F20X² | 0 | 69 | 19 | 12 |
| 5 | Mordenite (HCZM 20)³ | 57 | 21 | 21 | 1 |
| 6 | Zeolite Beta¹ | 0 | 67 | 22 | 11 |
| 7 | H-USY (CRV3014H)¹ | 0 | 81 | 13 | 6 |
| 8 | HCZE7 (Erionite)³ | 79 | 11 | 9 | 1 |
| 9 | Cha 30 (Chabazite)³ | 32 | 38 | 29 | 1 |
| 10 | CBV 3024E (ZSM-5)¹ | 0 | 71 | 20 | 9 |

¹Available from Zeolyst, Valley Forge, PA
²Available from EP Engineered Clays Corporation, Jackson, MS
³Clariant Corporation, Pasadena, TX

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the processes, machines, means, methods, and/or steps described in the specification. As one of the ordinary skill in the art will readily appreciate from the present disclosure, processes, machines, means, methods, and/or steps, presently existing or later to be developed that perform substantially the same function or achieve substantially the same result as the corresponding embodiments described herein, may be utilized according to the present disclosure. Accordingly, the appended claims are intended to include within their scope such processes, machines, means, methods, and/or steps.

What is claimed is:

1. A process comprising:
a) adding a feed stream comprising one or more propylene polyols, hydrogen, and optionally water, to a catalytic conversion reaction zone in the presence of a first solid acid catalyst component and a hydrogenation catalyst component to form a first reaction mixture; and
b) reacting the first reaction mixture at a temperature in the range of from 20° ° C. to 600° C., a pressure in the range of from 100 psig (689 kPag) to 1,500 psig (10,340 kPag), or a combination thereof, to form a hydrogenation product stream comprising a propanol component.

2. The process of claim 1, wherein the feed stream is added to the catalytic conversion reaction zone at a weight hourly space velocity in the range of from 0.1 h⁻¹ to 100 h⁻¹.

3. The process of claim 1, wherein the one or more propylene polyols comprise propylene glycol, di-propylene glycol, tri-propylene glycol, tetra-propylene glycol, or a combination thereof.

4. The process of claim 1, wherein the catalytic conversion product comprises 0 wt % to 90 wt % water and 10 wt % to 100 wt % organics other than water, wherein weight percentages are based on the total weight of the catalytic conversion product.

5. The process of claim 4, wherein the organics other than water comprise 1-propanol in the range of from 50 wt % to 90 wt % and other C₃ hydrocarbons in the range of from 10 wt % to 50 wt %, wherein weight percentages are based on the total weight of the organics other than water.

6. The process of claim 1, wherein the catalytic conversion product stream further comprises a propylene precursor component.

7. The process of claim 6, wherein the propylene precursor component comprises 1-propanol, 2-propanol, propionaldehyde, acetone, C₃ dioxanes, C₃ dioxolanes, propylene glycol, hydroxyacetone, or a combination thereof.

8. The process of claim 1, further comprising adding at least a portion of the catalytic conversion product to the catalytic conversion reaction zone.

9. The process of claim 1, wherein the first solid acid catalyst component comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, one or more polystyrene sulfonate acidic resins, sulfonate functionalized support, tethered organic sulfonic acids, acidic clays, or a combination thereof.

10. The process of claim 1, wherein the hydrogenation catalyst component comprises sulfided NiMo/Al₂O₃, sulfided CoMo/Al₂O₃, Ni/SiO₂, Ni/Al₂O₃, Raney Ni, Cu/SiO₂, Cu/Al₂O₃, Pd/SiO₂, Pd/Al₂O₃, Pd/C, Pt/SiO₂, Pt/Al₂O₃, Ru/C, In₂O₃ In₂O₃/Al₂O₃, In₂O₃/SiO₂, or a combination thereof.

11. The process of claim 1, wherein:
a) the first solid acid catalyst component is a first discrete catalyst, and the hydrogenation catalyst component is a second discrete catalyst; or
b) a hybrid catalyst comprises the first solid acid catalyst component and the hydrogenation catalyst component.

12. The process of claim 1, further comprising:
a) adding the catalytic conversion product stream to a dehydration reaction zone in the presence of a second solid acid catalyst to form a second reaction mixture; and
b) reacting the second reaction mixture at a temperature in the range of from 20° C. to 600° C., a pressure in the range of from 15 psig (103 kPag) to 500 psig (689 kPag), or a combination thereof, to form a dehydration product stream comprising propylene.

13. The process of claim 12, wherein the catalytic conversion product stream is added to the dehydration reaction zone at a weight hourly space velocity in the range of from 0.1 h⁻¹ to 100 h⁻¹.

14. The process of claim 12, wherein the second solid catalyst component comprises a zeolite component, an alumina silicate component, aluminum phosphate, zirconium sulfate, titanium sulfate, supported phosphoric acid, one or more supported tungsten oxides, supported tungstosilicic acid, supported phosphomolybdic acid, aluminum oxide, niobium oxide, or a combination thereof.

15. The process of claim 1, further comprising adding an organic waste stream, comprising one or more propylene polyols and a first content of one or more impurities harmful to the first solid acid catalyst component and/or the hydrogenation catalyst component, to a guard reaction zone to form the feed stream comprising one or more propylene polyols.

16. The process of claim 15, wherein the impurities comprise amines, urethane, amides, other nitrogen containing hydrocarbons, organic bases, caustic, or a combination thereof.

17. A process comprising:
a) adding a feed stream comprising one or more propylene polyols, hydrogen, and optionally water, to a catalytic conversion reaction zone in the presence of a first solid acid catalyst component and a hydrogenation catalyst component to form a first reaction mixture;
b) reacting the first reaction mixture at a temperature in the range of from 20° C. to 600° C., a pressure in the range of from 100 psig (689 kPag) to 1,500 psig (10,340 kPag), or a combination thereof, to form a catalytic conversion product stream comprising a propanol component; and
c) adding the catalytic conversion product to a first distillation column to produce a catalytic conversion product overhead stream and a catalytic conversion product bottoms stream and withdrawing the catalytic conversion overhead product stream as a first conversion product.

18. The process of claim 17, further comprising adding at least a portion of the first conversion product to the catalytic conversion reaction zone.

19. The process of claim 17, further comprising:
a) adding the first conversion product to a dehydration reaction zone in the presence of a second solid acid catalyst to form a second reaction mixture; and
b) reacting the second reaction mixture at a temperature in the range of from 20° ° C. to 600° C., a pressure in the range of from 15 psig (103 kPag) to 500 psig (689 kPag), or a combination thereof, to form a dehydration product stream comprising propylene.

20. The process of claim 19, further comprising:
a) withdrawing the dehydration product as a first propylene-containing product; or
b) routing the dehydration product to a second distillation column to produce a dehydration product overhead stream and a dehydration product bottoms stream and withdrawing the dehydration overhead product as a second propylene-containing product.
